(19) United States
(12) Patent Application Publication
(10) Pub. No.: US 2015/0293345 A1
(43) Pub. Date: Oct. 15, 2015

(54) MAGNIFICATION LOUPE WITH DISPLAY SYSTEM

(71) Applicant: ORANGEDENTAL GMBH & CO. KG, Biberach (DE)

(72) Inventors: Ludwig Laxhuber, Herrsching (DE); Stefan Kaltenbach, Rebstein (CH)

(21) Appl. No.: 14/443,961

(22) PCT Filed: Nov. 12, 2013
(86) PCT No.: PCT/EP2013/073543

§ 371 Date: May 19, 2015

(30) Foreign Application Priority Data
Nov. 19, 2012 (EP) 12193262.8

(51) Int. Cl. G02B 25/00; G02B 25/02; A61B 19/00; G02B 27/01

(52) U.S. Cl. CPC G02B 25/005; G02B 27/017; G02B 25/004; G02B 25/02; A61B 19/5212; A61B 19/5225; G02B 2027/0178; A61B 2019/5219

(57) ABSTRACT

The present invention relates to optical instruments, in particular to magnification loupes, such as those worn by dentists and surgeons. The magnification loupes are equipped with one or more cameras and with one or more displays and/or with an audio assembly. The invention also relates to a system comprising such magnification loupes and a computer system.

The present invention relates to optical instruments, in particular to magnification loupes, such as those worn by dentists and surgeons. The magnification loupes are equipped with one or more cameras and with one or more displays and/or with an audio assembly. The invention also relates to a system comprising such magnification loupes and a computer system.

### BACKGROUND AND TECHNICAL PROBLEMS UNDERLYING THE PRESENT INVENTION

Magnification loupes used by dentists or surgeons typically comprise a spectacle frame with two spectacle lenses and two magnifying optical systems (one for each eye) that are fixed to the spectacle lenses. In addition, magnification loupes often comprise a light source so that the operating field is illuminated in surgical procedures or the patient's mouth is illuminated in dental examinations.

A practitioner or dentist treating a patient often needs to retrieve or update patient data which is usually stored in the internal computer network of the doctor's office or dentist's office. However, it is cumbersome for the practitioner or dentist to interrupt the treatment and to operate a computer only to obtain or update information on a patient.

The present inventors found out that magnification loupes can be advantageously combined with an audio assembly and/or with displays so that information can be presented to the surgeon or dentist acoustically (via the audio assembly) or optically (via displays) without interruption of the treatment procedure. Moreover, the surgeon or dentist can use the audio assembly to update patient data.

The display system provides additional advantages when combined with a camera system. For example, the combination of display system and cameras makes it possible to construct magnification loupes without magnifying optical systems and without a light source. In addition, the present invention makes it possible to transmit images of a treatment, as seen by the surgeon or dentist, to a computer system for documentation or educational purposes.

The present inventors constructed novel magnification loupes that address the above needs. The novel magnification loupes are described in the appended claims and in the following description.

The above overview does not necessarily describe all problems solved by the present invention.

### SUMMARY OF THE INVENTION

In a first aspect the present invention relates to a magnification loupe (1), comprising (a) a spectacle frame (10) comprising a right temple (11) and a left temple (12); (b) a right camera (21) mounted to the right temple (11), said right camera (21) having a viewing direction which is facing towards a point which lies in the viewing direction of a person wearing the magnification loupe (1) and which lies about 20 cm to about 60 cm in front of the right eye of a person wearing the magnification loupe (1); (c) a left camera (22) mounted to the left temple (12), said left camera (22) having a viewing direction which is facing towards a point which lies in the viewing direction of a person wearing the magnification loupe (1) and which lies about 20 cm to about 60 cm in front of the left eye of a person wearing the magnification loupe (1); (d) a right display (31) which is structured and configured to display an image recorded by the right camera (21) in real-time; said right display (31) being mounted to the spectacle frame (10) in such a manner that said right display (31) is in front of the right eye of a person wearing the magnification loupe (1); and (e) a left display (32) which is structured and configured to display an image recorded by the left camera (22) in real-time; said left display (32) being mounted to the spectacle frame (10) in such a manner that said left display (32) is in front of the left eye of a person wearing the magnification loupe (1).

In a second aspect the present invention relates to a magnification loupe (1), comprising (a) a spectacle frame (10); (b) a magnifying optical system (40); and (c) an audio assembly (50).

In a third aspect the present invention relates to a system comprising (i) the magnification loupe (1) according to any one of the first or second aspect; and (ii) a computer system (70).

This summary of the invention does not necessarily describe all features of the present invention. Other embodiments will become apparent from a review of the ensuing detailed description.

### BRIEF DESCRIPTION OF THE DRAWINGS

FIG. 1 shows a top plan view of a magnification loupe according to one embodiment of the present invention. The arrow with the dashed line shows a light ray emanating from an object viewed by the person wearing the magnification loupe (the person is not shown). The viewing direction of the person wearing the magnification loupe is towards the bottom of the figure.

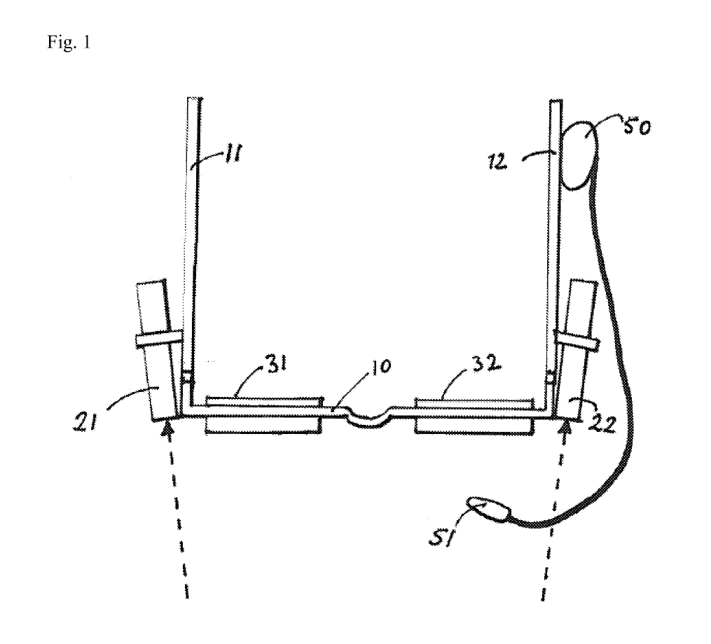

FIG. 2 shows a top plan view of a magnification loupe according to another embodiment of the present invention. The arrow with the dashed line shows a light ray emanating from an object viewed by the person wearing the magnification loupe (the person is not shown). The three arrows with the solids lines signify light beams emanating from light source (60). The viewing direction of the person wearing the magnification loupe is towards the bottom of the figure.

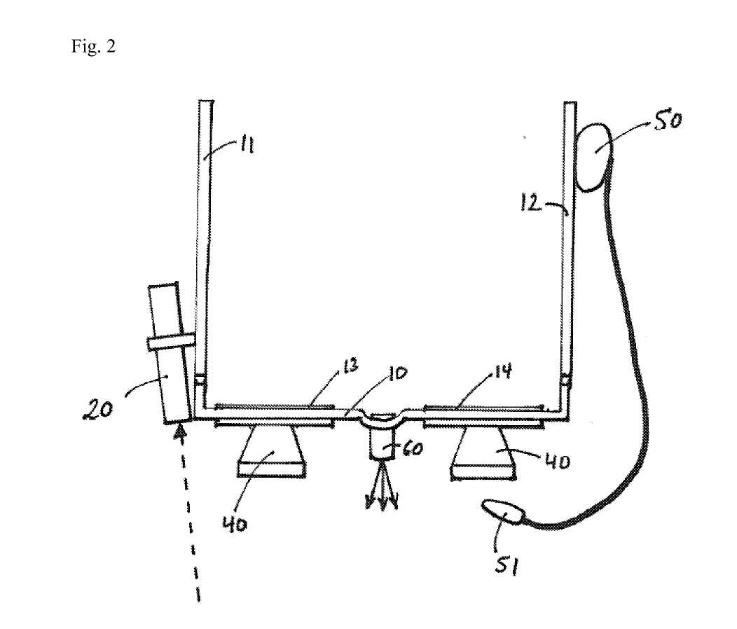

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents (for example: patents, patent applications, scientific publications, manufacturer's specifications, instructions, etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

The term “about” when used in connection with a numerical value is meant to encompass numerical values within a range having a lower limit that is 5% smaller than the indicated numerical value and having an upper limit that is 5% larger than the indicated numerical value.

In the context of the present application, the term “user” refers to a person wearing the magnification loupe of the invention.

### EMBODIMENTS OF THE INVENTION

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

In a first aspect the present invention is directed to a magnification loupe (1), comprising

(a) a spectacle frame (10) comprising a right temple (11) and a left temple (12);

(b) a right camera (21) mounted to the right temple (11), said right camera (21) having a viewing direction which is facing towards a point which lies in the viewing direction of a person wearing the magnification loupe (1) and which lies about 20 cm to about 60 cm in front of the right eye of a person wearing the magnification loupe (1);

(c) a left camera (22) mounted to the left temple (12), said left camera (22) having a viewing direction which is facing towards a point which lies in the viewing direction of a person wearing the magnification loupe (1) and which lies about 20 cm to about 60 cm in front of the left eye of a person wearing the magnification loupe (1);

(d) a right display (31) which is structured and configured to display an image recorded by the right camera (21) in real-time; said right display (31) being mounted to the spectacle frame (10) in such a manner that said right display (31) is in front of the right eye of a person wearing the magnification loupe (1); and

(e) a left display (32) which is structured and configured to display an image recorded by the left camera (22) in real-time; said left display (32) being mounted to the spectacle frame (10) in such a manner that said left display (32) is in front of the left eye of a person wearing the magnification loupe (1).

In the first aspect of the present invention, a magnification of the observed object or area is achieved by the cameras (21, 22) and the displays (31, 32). Thus, in this aspect of the present invention, there is no need for a separate magnifying optical system (40). More specifically, the cameras (21, 22) record images of the observed object or area and display these images in real-time on the displays (31, 32). The user can adjust various parameters of the images that are shown on the displays (31, 32). In particular, the user can adjust the magnification, the contrast and/or the brightness of the images shown on the display. In particular preferred embodiments, these adjustments can be made via voice control.

In particularly preferred embodiments, the cameras (21, 22) used in the first aspect are sensitive cameras that are designed to work in low light conditions. By employing such sensitive cameras it is possible to dispense with light sources. Magnification loupes of the prior art typically use a light source that is mounted to the spectacle frame.

The displays (31, 32) can be mounted to the spectacle frame (10) in such a manner that they replace spectacle lenses, which are normally a component of magnification loupes. It is also possible that the spectacle lenses (13, 14) are only partially replaced by the displays (31, 32). For example, it is considered within the present invention, that the lower half of each spectacle lens (13, 14) is replaced by a display (31, 32) so that the user can watch the images recorded by the cameras (21, 22) when looking down (i.e. to the displays) and has a normal view on his surroundings when looking up (i.e. through the spectacle lenses (13, 14). It is possible that this arrangement of spectacle lenses (13, 14) and displays (31, 32) is reversed; the user would then have a normal view when looking down and would see images recorded by the cameras (21, 22) when looking up. In a variant of the aforementioned embodiments, the displays (31, 32) do not replace the spectacle lenses (13, 14) but are present in addition to the spectacle lenses. In this variant, the user looks through the spectacle lenses (13, 14) to watch the images on the displays (31, 32).

The display can also be used to show additional information to the user. For example, information on the currently treated patient may be retrieved from a computer file and displayed on one or both of the displays (31, 32).

In all embodiments of the present invention that use spectacle lenses (13, 14), these spectacle lenses (13, 14) need not be corrective lenses; they may be simple glass panes. However, for users that need corrective lenses, it is also possible to use such corrective lenses as the right and left spectacle lenses (13, 14) of the magnification loupe (1).

In preferred embodiments of the first aspect, at least one of the right camera (21) or the left camera (22) is structured and configured to wirelessly transmit images recorded by said right camera (21) or said left camera (22) to a monitor or to a computer or to a further display in real-time. These embodiments provide the advantage that a second person (e.g. another practitioner, dentist or surgeon; or a dental assistant) can observe what is seen by the user. For example, said second person can watch a medical treatment of a patient performed by the user. In addition, the images transmitted to a computer can be stored for documentation or educational purposes.

In preferred embodiments of the first aspect, the magnification loupe (1) further comprises an audio assembly (50). Preferably, the audio assembly (50) is structured and configured to wirelessly receive and transmit audio signals. Advantageously, the audio assembly comprises a microphone (51) which is structured and configured to wirelessly transmit audio signals to a computer system. The microphone (51) can be connected to the audio assembly (50) via a bendable wire so that the microphone (51) can be positioned close to the user's mouth. The audio assembly (50) can be mounted to one of the temples (11, 12). In a further advantageous embodiment, the audio assembly comprises one or more loudspeakers (52) (preferably one or two loudspeakers), which are structured and configured to provide the person wearing the magnification loupe (1) with acoustic information (audio signals). In embodiments in which two loudspeakers (52) are used, one loudspeaker is positioned near (or in) the right ear of the person and the other loudspeaker is positioned near (or in) the left ear of the person.

In a second aspect the present invention is directed to a magnification loupe, comprising (a) a spectacle frame (10); (b) a magnifying optical system (40); and (c) an audio assembly (50).

The spectacle frame (10) typically comprises a right temple (11) and a left temple (12). The magnification loupe (1) of the second aspect may additionally comprise a right spectacle lens (13) and a left spectacle lens (14). The spectacle lenses (13, 14) need not be corrective lenses; they may be simple glass panes. However, for users that need corrective lenses, it is also possible to use such corrective lenses as the right and left spectacle lenses (13, 14) of the magnification loupe (1). The magnifying optical system (40) can be fixed to the right spectacle lens (13) and to the left spectacle lens (14).

In preferred embodiments of the second aspect, the magnification loupe further comprises: (d) a light source (60) structured and configured to emit light in the visible spectrum, wherein the direction of the emitted light is parallel to the viewing direction of a person wearing the magnification loupe.

In further preferred embodiments of the second aspect, the magnification loupe further comprises: (e) at least one camera (20) mounted to a temple of the spectacle frame, said at least one camera (20) having a viewing direction which is facing towards a point which lies in the viewing direction of a person wearing the magnification loupe (1) and which lies about 20 cm to about 60 cm in front of the eyes of a person wearing the magnification loupe (1). In preferred embodiments of the second aspect, the camera (20) is a sensitive camera that is designed to work in low light conditions.

Preferably, said at least one camera (20) is structured and configured to wirelessly transmit images recorded by said at least one camera (20) to a monitor or to a computer or to a display in real-time. This embodiment provides the advantage that a second person (e.g. another practitioner, dentist or surgeon; or a dental assistant) can observe what is seen by the user. For example, said second person can watch a medical treatment of a patient performed by the user. In addition, the images transmitted to a computer can be stored for documentation or educational purposes.

In preferred embodiments of the second aspect, the audio assembly (50) is structured and configured to wirelessly receive and transmit audio signals. Advantageously, the audio assembly (50) comprises a microphone (51) which is structured and configured to wirelessly transmit audio signals to a computer system (70). The microphone (51) can be connected to the audio assembly (50) via a bendable wire so that the microphone (51) can be positioned close to the user's mouth. The audio assembly (50) can be mounted to one of the temples (11, 12). In a further advantageous embodiment, the audio assembly comprises one or more loudspeakers (52) (preferably one or two loudspeakers), which are structured and configured to provide the person wearing the magnification loupe (1) with acoustic information (audio signals). In embodiments in which two loudspeakers (52) are used, one loudspeaker is positioned near (or in) the right ear of the person and the other loudspeaker is positioned near (or in) the left ear of the person.

As indicated above for the first and the second aspect of the invention, each camera (20, 21, 22) has a viewing direction which is facing towards a point which lies in the viewing direction of a person wearing the magnification loupe (1) (i.e. the user) and which lies about 20 cm to about 60 cm in front of the eyes (right eye and/or left eye) of the user. The reason for this is that the cameras (20, 21, 22) should be oriented towards an object which is viewed by the user.

Furthermore, in typical applications of the first and second aspect, the user is a practitioner, a dentist or a surgeon looking at his patient. A practitioner, dentist or surgeon treating a patient will typically look at an area to be treated from a distance of between 20 cm to 60 cm. Therefore, the distance between the eye of the user and the observed point (object or area) will mostly lie in this range. More preferably, said point lies about 30 cm to about 60 cm, even more preferably about 40 cm to about 50 cm in front of right eye or left eye of the user. In preferred embodiments, the cameras (20, 21, 22) are movably mounted to the temples (11, 12) of the spectacle frame (10) to allow an adjustment of the cameras (20, 21, 22) to various distances in the above-mentioned ranges. In some embodiments, the cameras may be adjusted by small electric motors.

In particularly advantageous embodiments of the first and second aspect, the magnification loupes of the present invention can be used by a dentist looking at the open mouth of his patient.

In other embodiments of the first and second aspect, each camera has a viewing direction which is parallel to the viewing direction of a person wearing the magnification loupe. This parallel orientation is especially useful, when the user wants to look at objects that are further away, e.g. at a distance larger than 2 m.

In a third aspect the present invention is directed to a system comprising (i) the magnification loupe (1) according to any one of the first or second aspect; and (ii) a computer system (70).

In preferred embodiments of the third aspect, the magnification loupe comprises a microphone (51) which is structured and configured to wirelessly transmit audio signals to said computer system (70).

In preferred embodiments of the third aspect, the computer system is structured and configured to run speech recognition software. Preferably, said speech recognition software is structured and configured to translate audio signals recorded by the microphone (51) and transmitted to the computer system (70) into program commands.

In embodiments employing speech recognition software, the following applications are contemplated within the context of the present invention: Speech recognition software running on the computer system can convert the audio signals into program code or into text information. By using such a system, it is possible that the user opens a computer file containing information on the currently treated patient; views said information on an external monitor, an external display, or on one or both of the displays (31, 32) mounted to the magnification loupe (1) of the first aspect; adds further patient-related information to the file; and saves the modified file. It is also possible that the person wearing the magnification loupe (1) receives acoustic information from a computer system (70) via the loudspeaker(s) (52). For example, the user can open a computer file pertaining to the currently treated patient by means of voice control and can give a command to the computer system to read aloud (selected) information from the file.

It is also contemplated within the context of the present invention that audio signals are wirelessly transmitted from the person speaking into the microphone (51) to another person in another room (e.g. to a doctor's receptionist, to a dental assistant, or to a medical assistant). In some embodiments, the person may use the microphone (51) and loudspeaker(s) (52) to communicate with another person in another room (e.g. with a doctor's receptionist, with a dental assistant, or with a medical assistant).

The cameras (20, 21, 22), the displays (31, 32), the audio assembly (50) (including a microphone (51) and loudspeaker (52)), and the light source (60) can be powered with electric energy by an energy storage component (90). The energy storage component (90) can be positioned in a compartment within the right temple (11) and/or the left temple (12). Suitable energy storage components (90) are rechargeable batteries (91), for example polymer batteries, such as lithium ion polymer batteries.

### LISTING OF REFERENCE NUMERALS

1 magnification loupe
10 spectacle frame
11 right temple
12 left temple
13 right spectacle lens
14 left spectacle lens
20 camera
21 right camera
22 left camera
31 right display
32 left display
40 magnifying optical system
50 audio assembly
51 microphone
52 loudspeaker
60 light source (structured and configured to emit light in the visible spectrum)
70 computer system
90 energy storage component
91 rechargeable battery
92 polymer battery

1. A magnification loupe comprising:
(a) a spectacle frame comprising a right temple and a left temple;
(b) a right camera mounted to the right temple, said right camera having a viewing direction which is facing towards a point which lies in the viewing direction of a person wearing the magnification loupe and which lies about 20 cm to about 60 cm in front of a right eye of the person wearing the magnification loupe;
(c) a left camera mounted to the left temple, said left camera having a viewing direction which is facing towards a point which lies in the viewing direction of the person wearing the magnification loupe and which lies about 20 cm to about 60 cm in front of a left eye of the person wearing the magnification loupe;
(d) a right display which is structured and configured to display an image recorded by the right camera in real-time; said right display being mounted to the spectacle frame in such a manner that said right display is in front of the right eye of a person wearing the magnification loupe; and
(e) a left display which is structured and configured to display an image recorded by the left camera real-time; said left display being mounted to the spectacle frame in such a manner that said left display in front of the left eye of a person wearing the magnification loupe.

2. The magnification loupe according to claim 1, wherein at least one of the right camera or the left camera is structured and configured to wirelessly transmit images recorded by said right camera or said left camera to a monitor or to a computer or to a further display in real-time.

3. The magnification loupe according to claim 1, further comprising an audio assembly.

4. A magnification loupe comprising:
(a) a spectacle frame;
(b) a magnifying optical system; and
(c) an audio assembly.

5. The magnification loupe according to claim 4, further comprising:
(d) a light source structured and configured to emit light in the visible spectrum, wherein a direction of the emitted light is parallel to a viewing direction of a person wearing the magnification loupe.

6. The magnification loupe according to claim 4, further comprising:
(e) at least one camera, said at least one camera having a viewing direction which is facing towards a point which lies in the viewing direction of a person wearing the magnification loupe and which lies about 20 cm to about 60 cm in front of the eyes of the person wearing the magnification loupe.

7. The magnification loupe according to claim 6, wherein said at least one camera is structured and configured to wirelessly transmit images recorded by said at least one camera to a monitor or to a computer or to a display in real-time.

8. The magnification loupe according to claim 3, wherein the audio assembly is structured and configured to wirelessly receive and transmit audio signals.

9. The magnification loupe according to claim 8, wherein the audio assembly comprises a microphone which is structured and configured to wirelessly transmit audio signals to a computer system.

10. The magnification loupe according to claim 8, wherein the audio assembly comprises one or more loudspeakers, which are structured and configured to provide a person wearing the magnification loupe with acoustic information.

11. A system comprising:
(i) the magnification loupe according to claim 1; and
(ii) a computer system.

12. The system according to claim 11, wherein said magnification loupe comprises a microphone which is structured and configured to wirelessly transmit audio signals to said computer system.

13. The system according to claim 11, wherein the computer system is structured and configured to run speech recognition software.

14. The system according to claim 13, wherein said speech recognition software is structured and configured to translate audio signals recorded by the microphone and transmitted to the computer system into program commands.

15. The magnification loupe according to claim 2, further comprising an audio assembly.

16. A system comprising:
(i) the magnification loupe according to claim 15; and
(ii) a computer system,
wherein said magnification loupe comprises a microphone which is structured and configured to wirelessly transmit audio signals to said computer system.

17. The system according to claim 16, wherein the computer system is structured and configured to run speech recognition software, and wherein said speech recognition software is structured and configured to translate audio signals recorded by the microphone and transmitted to the computer system into program commands.

18. A system comprising:
(i) the magnification loupe according to claim 4; and
(ii) a computer system,
wherein said magnification loupe comprises a microphone which is structured and configured to wirelessly transmit audio signals to said computer system.

19. A system comprising:
the magnification loupe according to claim 9, wherein the computer system is structured and configured to run speech recognition software.

20. The system according to claim 19, wherein said speech recognition software is structured and configured to translate audio signals recorded by the microphone and transmitted to the computer system into program commands.
